(19) United States
(12) Patent Application Publication
(10) Pub. No.: US 2025/0265743 A1
(43) Pub. Date: Aug. 21, 2025

(54) METHODS AND SYSTEMS FOR PROVIDING AN IMAGE ACQUISITION INFORMATION OF A MEDICAL IMAGE

(71) Applicant: Siemens Healthineers AG, Forchheim (DE)

(72) Inventors: Yoshihisa SHINAGAWA, Downingtown, PA (US); Enlin QIAN, Cliffside, NJ (US); Kritika IYER, Conshohocken, PA (US); Gerardo HERMOSILLO VALADEZ, West Chester, PA (US)

(73) Assignee: Siemens Healthineers AG, Forchheim (DE)

(21) Appl. No.: 19/054,970

(22) Filed: Feb. 17, 2025

(30) Foreign Application Priority Data
Feb. 19, 2024 (DE) 10 2024 201 496.6

(51) Int. Cl. G06T 11/00; G06T 7/00; G16H 30/40

(52) U.S. Cl. CPC G06T 11/008; G06T 7/0016; G06T 11/006; G16H 30/40; G06T 2200/24; G06T 2207/10081; G06T 2207/10088; G06T 2207/20081; G06T 2207/20221; G06T 2210/41; G06T 2211/441

(57) ABSTRACT

Computer-implemented methods and systems for providing an image acquisition information of a medical image are provided. The methods and systems implement a plurality of steps. One step is directed to receive the medical image. Another step is directed to transform the medical image (or data of the medical image) into a frequency domain to obtain a k-space image. Another step is directed to determine the image acquisition information by applying a trained function to the k-space image (or to data of the k-space image). Another step is directed to provide the image acquisition information.

### CROSS-REFERENCE TO RELATED APPLICATION(S)

The present application claims priority under 35 U.S.C. § 119 to German Patent Application No. 10 2024 201 496.6, filed Feb. 19, 2024, the entire contents of which is incorporated herein by reference.

### FIELD

One or more example embodiments relate to methods and systems for providing image acquisition information of medical images. One or more example embodiments relate to systems and methods for deriving image acquisition information from pixel or voxel data of medical images. One or more example embodiments relate to the usage of the image acquisition information for processing the medical image, in particular, for deriving a medical diagnosis.

### RELATED ART

Automation of workflows is still a challenge in the processing of medical images for arriving at a possible diagnosis of a patient. Thereby, image processing starts with tasks which might appear trivial at first sight such as the selection of appropriate displaying settings or the placement of the image data on a displaying screen of the reviewing physician. It goes on with more subtle decisions such as the selection of the correct image processing tools, in particular, computer aided detection (CAD) tools, the selection of auxiliary data, the routing of data to the competent physician, the selection of appropriate reporting templates for creating the final medical report, and so forth.

Automating these and other steps is difficult as these steps crucially depend on the underlying medical image. To put it differently, different images may require a very different treatment in a reading and reporting workflow. The characteristics of the medical image may be summed up in the image acquisition information indicating, e.g., the imaging modality and the settings used, or the body part displayed.

One option could be to read this information from the documentation available for the medical images. One issue with this approach is that there often is too little information documented for the image acquisition parameters which would allow to make an educated decision regarding automated processing steps. Further, regarding the image studies to be automatically processed there often is huge variety. This can come from different available acquisition and/or reconstructions technologies, different modalities, or even different preferences in the acquisition.

This is even more important because the type of the image data and the image acquisition parameters such as the CT imaging protocol or the MR sequence are decisive for many tasks which can be potentially automated. This concerns potential post-processing steps (e.g., convolution kernels), image enhancement steps (applying the correct window, e.g., bone), tool selection (e.g., lung CAD for lung-related image data), the hanging order, or prior selection. The latter is of particular importance in cases where a longitudinal observation of patients is required to determine the development of certain diseases like multiple sclerosis or tumor follow ups.

What is more, in the clinical reality, the documentation of imaging parameters and imaged organs is not only scarce but oftentimes also not existent or flat wrong. Due to the time pressure in the clinical routine, there often are dummy entries in the protocols which cannot be used.

As a consequence, any straight-forward automation which is (only) based on what is expressively documented in a patient case often leads to inappropriate results. This not only consumes system resources but may lead to extra work at the side of the users-which is completely contrary to the original intention of automizing the workflow in radiology reading and reporting.

To overcome these difficulties, it was proposed to automatically extract relevant image acquisition information by applying machine-learned function to the medical images. For instance, the reference of van der Voort et al., DeepDicomSort: An Automatic Sorting Algorithm for Brain Magnetic Resonance Imaging Data, in Neuroinformatics, 2021 January, 19(1):159-184, doi: 10.1007/s12021-020-09475-7 proposes using a convolutional neural network (CNN) that automatically recognizes eight different brain magnetic resonance imaging (MRI) scan types based on visual appearance.

### SUMMARY

While such methods generally perform well for sorting tasks, the inventors have found that the accuracy for correctly predicting subtle differences which matter in workflow automation is not sufficient. Further, suchlike methods do not generalize very well to unseen anatomies.

Accordingly, embodiments of the invention improve the processing of medical images for obtaining a medical diagnosis. In particular, embodiments of the invention improve the provision of image acquisition information of a medical image, such as a subsequent usage in the processing of the medical image aiming at obtaining a medical diagnosis based on the medical image.

This is solved by a method for providing an image acquisition information of a medical image, a system for providing an image acquisition information of a medical image, corresponding computer-program products, and corresponding computer-readable storage media according to the main claims. Alternative and/or preferred embodiments are object of the dependent claims.

In the following, the technical solution according to the present invention is described with respect to the claimed apparatuses as well as with respect to the claimed methods. Features, advantages, or alternative embodiments described herein can likewise be assigned to other claimed objects and vice versa. In other words, claims addressing the inventive method can be improved by features described or claimed with respect to the apparatuses. In this case, e.g., functional features of the method are embodied by objective units or elements of the apparatus.

### BRIEF DESCRIPTION OF THE DRAWINGS

Characteristics, features and advantages, as well as the manner they are achieved, become clearer and more understandable in the light of the following description of embodiments, which will be described in detail with respect to the figures. This following description does not limit the invention on the contained embodiments. Same components, parts or steps can be labeled with the same reference signs in different figures. In general, the figures are not drawn to scale. In the following:

FIG. 1 schematically depicts a system for providing an image acquisition information and/or a representation of a medical image according to an embodiment,

FIG. 2 schematically depicts a method for providing an image acquisition information of a medical image according to an embodiment,

FIG. 3 schematically depicts data flows in a method for providing an image acquisition information of a medical image according to an embodiment,

FIG. 4 schematically depicts a method for providing a representation of a medical image according to an embodiment,

FIG. 5 schematically depicts data flows in a method for providing a representation of a medical image according to an embodiment,

FIG. 6 schematically depicts a graphical user interface for displaying a representation of a medical image according to an embodiment, and

FIG. 7 schematically depicts a trained function for determining an image acquisition information of a medical image according to an embodiment.

### DETAILED DESCRIPTION

The technical solution will be described both with regard to methods and systems for providing an image acquisition information of a medical image and also with regard to methods and systems for providing a trained function configured for extracting the image acquisition information from the medical image. Features and alternate forms of embodiments of data structures and/or functions for methods and systems for providing trained functions can be transferred to analogous data structures and/or functions for methods and systems for providing the image acquisition information. Analogous data structures can, in particular, be identified by using the prefix “training”. Furthermore, the prediction functions used in methods and system for providing information can, in particular, have been adjusted and/or trained and/or provided by methods and systems for adjustment of trained functions.

According to an aspect, a computer-implemented method for providing an image acquisition information of a medical image is provided. The method comprises a plurality of steps. One step is directed to receiving the medical image. Another step is directed to transforming the medical image (or data of the medical image) into the frequency domain so as to obtain a k-space image. Another step is directed to determining the image acquisition information by applying a trained function to the k-space image (or to data of the k-space image). Another step is directed to provide the image acquisition information.

The medical image may be a two-dimensional image. Further, the medical image data set may be a three-dimensional image. Further, the medical image may be a four-dimensional image, where there are three spatial and one time-like dimensions. Further, the medical image data set may comprise a plurality of individual medical images.

The medical image comprises image data, for example, in the form of a two-or three-dimensional array of pixels or voxels. Such arrays of pixels or voxels may be representative of color, intensity, absorption or other parameters as a function of two or three-dimensional position, and may, for example, be obtained by suitable processing of measurement signals obtained by a medical imaging modality or image scanning facility.

The medical image may depict a body part of a patient. Accordingly, it may contain two or three-dimensional image data of the patient's body part. The medical image may be representative of an image volume or a cross-section through the image volume. The patient's body part may be comprised in the image volume.

A medical imaging modality corresponds to a system used to generate or produce medical image data. For example, a medical imaging modality may be a computed tomography system (CT system), a magnetic resonance system (MR system), an angiography (or C-arm X-ray) system, a positron-emission tomography system (PET system) or the like. Specifically, computed tomography is a widely used imaging method and makes use of “hard” X-rays produced and detected by a spatially rotating instrument. The resulting attenuation data (also referred to as raw data) is processed by a computed analytic software producing detailed images of the internal structure of the patient's body parts. The produced sets of images are called CT-scans which may constitute multiple series of sequential images to present the internal anatomical structures in cross sections perpendicular to the axis of the human body. Magnetic Resonance Imaging (MRI), to provide another example, is an advanced medical imaging technique which makes use of the effect magnetic field impacts on movements of protons. In MRI machines, the detectors are antennas, and the signals are analyzed by a computer creating detailed images of the internal structures in any section of the human body.

A medical image may comprise a plurality of image slices. The slices respectively show a cross-sectional view of the image volume. The slices may comprise a two-dimensional array of pixels or voxels as image data. The arrangement of slices in the medical image data set may be determined by the imaging modality or by any post-processing scheme used. Further, slices may artificially be defined in the imaging volume spanned by the medical image data set. Optionally, this may happen as a function of the image data comprised in the medical image data set in order to optimally pre-process the medical image data set for the ensuing diagnostic workflow.

Further, a medical image may comprise a plurality of image series each representing an individual scan of the patient. Each series may relate to a 2D or 3D imaged area.

The medical image may be stored in a standard image format such as the Digital Imaging and Communications in Medicine (DICOM) format and in a memory or computer storage system such as a Picture Archiving and Communication System

(PACS), a Radiology Information System (RIS), and the like. Whenever DICOM is mentioned herein, it shall be understood that this refers to the “Digital Imaging and Communications in Medicine” (DICOM) standard, for example according to the DICOM PS3.1 2020c standard (or any later or earlier version of said standard).

According to some examples, the medical image is received from a Picture Archiving and Communication System (PACS). Thereby, the PACS may store the medical images in a post-processed version after acquisition. The post-processed version may relate to readily viewable version in an image viewer and not to raw data. In particular, the medical image may not comprise any data in the frequency domain. In other words, the image data may (only) comprise “real” data.

The frequency domain may be seen as the mathematical space of spatial frequencies. Frequency domain and the real (Euclidean) space (in the space domain) are connected by the Fourier transform. Specifically, data may be transformed from the real space to the frequency domain by applying the 2D or 3D Fourier transform and from the frequency domain to the real space using the inverse or reciprocal Fourier transform.

Other words for frequency domain may be reciprocal space, frequency space or k-space or Fourier-space.

According to some examples, the medical image may be seen as the real image and the k-space image may be seen as the frequency domain image (of the medical image) or as the medical image transformed into the frequency domain.

The k-space image may have the same dimensions, that is, the same number of rows and/or columns as the medical image. The entries in the rows and/or columns may reflect the occurrence of spatial frequencies in the medical image. The entries may have real and imaginary components. It should be noted, that the k-space can have arbitrary sizes. However, making the k-space very large, e.g., beyond the highest frequencies that scanners can measure, is not meaningful as this does not increase the information content.

According to some examples, the step of transforming the medical image (or data of the medical image) into the frequency domain so as to obtain a k-space image may comprise applying a Fourier transform, in particular, a discrete Fourier transform, to the medical image. This may comprise decomposing a sequence of values, i.e., the pixels/voxels of the medical image, into components of different frequencies.

The trained function is configured (or has been trained) to extract image acquisition information relating to a medical image from a corresponding k-space image of the medical image. Applying the trained function may comprise providing the trained function and/or inputting the respective data (i.e., data of k-space image and/or data of the medical image) in the trained function.

In general, a trained function mimics cognitive functions that humans associate with other human minds. In particular, by training based on training data, the machine-learned function is able to adapt to new circumstances and to detect and extrapolate patterns, e.g., different scanners and manufactures. Other terms for machine-learned function, may be machine-learned function, trained machine learning model, trained mapping specification, mapping specification with trained parameters, function with trained parameters, algorithm based on artificial intelligence, or machine learned algorithm.

In general, parameters of a machine-learned function can be adapted via training. In particular, supervised training, semi-supervised training, unsupervised training, reinforcement learning and/or active learning can be used. Furthermore, representation learning (an alternative term is “feature learning”) can be used. In particular, the parameters of the machine-learned function can be adapted iteratively by several steps of training.

In particular, a trained function can comprise a neural network, a support vector machine, a decision tree and/or a Bayesian network, and/or the trained function can be based on k-means clustering, Q-learning, genetic algorithms and/or association rules. In particular, a neural network can be a deep neural network, a convolutional neural network or a convolutional deep neural network. Furthermore, a neural network can be an adversarial network, a deep adversarial network and/or a generative adversarial network.

The trained function may be generally configured to determine image acquisition information based on k-space images obtained by transforming the medical image into the frequency domain. For instance, the trained function may be configured to extract one or more features from the k-space and image and map/classify these features into a feature space associated with different image acquisition parameters for determining which acquisition information the k-space image indicates. Thus, the trained function may comprise a feature extractor and a classifier. In particular, the feature extractor and the classifier may be implemented as a neural network, in particular, a convolutional neural network, with some network layers trained to extract features and other network layers being trained to provide a classification according to the most likely image acquisition information.

According to some examples, the image acquisition information relates to an image acquisition procedure with a medical imaging modality with which image acquisition procedure the medical image has been acquired. According to some examples, the image acquisition information comprises a modality, an anatomy, and/or a procedure based on which the medical image has been acquired. According to some examples, the image acquisition information comprises one or more image acquisition parameters with which or based on which the medical image has been acquired, such as a type of the medical imaging modality used, settings of the medical imaging modality, or a type of the medical image data.

The inventors have recognized that, by transforming the medical image data into the frequency domain, the image acquisition information becomes better accessible for an automated feature extraction and classification of a trained function. With that, the image acquisition information may be obtained in a more secure manner especially in cases where this information is not documented in the available medical record of the patient and/or the meta-data of the medical image. In turn, this allows for a more efficient automation of the process directed to the provision of a medical diagnosis based on the medical image.

According to some examples, the step of transforming the medical image (or data of the medical image) into the frequency domain so as to obtain a k-space image comprises applying a Fast Fourier Transformation (FFT) to the medical image.

An FFT is an algorithm that computes the discrete Fourier transform of original data or its inverse. An FFT computes such transformations by matrix-defactorization. With that, a computationally efficient procedure may be used.

In principle, all suited trained functions may be used for obtaining the image acquisition information from the k-space image.

According to some examples, however, the trained function comprises at least one of: a convolutional neural network, a transformer network, and/or a FocalNet.

A convolutional neural network is a neural network that uses a convolution operation instead of general matrix multiplication in at least one of its layers (so-called “convolutional layer”). In particular, a convolutional layer performs a dot product of one or more convolution kernels with the convolutional layer's input data/image, wherein the entries of the one or more convolution kernel are the parameters or weights that are adapted by training. In particular, one can use the Frobenius inner product and the ReLU activation function. A convolutional neural network can comprise additional layers, e.g., pooling layers, fully connected layers, and normalization layers.

For an example for the general usability of convolutional neural networks for deriving image acquisition parameters, reference is made to van der Voort et al., DeepDicomSort: An Automatic Sorting Algorithm for Brain Magnetic Resonance Imaging Data, in Neuroinformatics, 2021 January, 19(1):159-184, doi: 10.1007/s12021-020-09475-7, the contents of which are herein included by reference in their entirety. While being applied on real image data in van der Voort et al., the inventors have recognized that the architecture may, in principle, also be applied to k-space images.

By using convolutional neural networks, input k-space images can be processed in a very efficient way, because a convolution operation based on different kernels can extract various image features, so that, by adapting the weights of the convolution kernel, the relevant frequency patterns can be readily found during training. Furthermore, based on the weight-sharing in the convolutional kernels, less parameters need to be trained, which prevents overfitting in the training phase and allows to have faster training or more layers in the network, improving the performance of the network.

A transformer network is a neural network architecture generally comprising an encoder, a decoder, or both an encoder and decoder.

In some instances, the encoders and/or decoders are composed of several corresponding encoding layers and decoding layers, respectively. Within each encoding and decoding layer is an attention mechanism. The attention mechanism, sometimes called self-attention, relates data elements (such as words or pixels) within a series of data elements to other data elements within the series.

The encoder, in particular, may be configured to transform the input k-space image into a numerical representation. The numerical representation may comprise a vector per input token (e.g., per image patch). The encoder may be configured to implement an attention mechanism so that each patch is affected by the other patches in the input. In particular, the encoder may be configured such that the representations resolve the desired output, i.e., the image acquisition information of the trained function.

The decoder, in particular, may be configured to transform an input into a sequence of output tokens. In particular, the decoder may be configured to implement a masked self-attention mechanism so that each vector of a token is affected only by the other tokens to one side of a sequence. Further, the decoder may be auto-regressive in the sense that intermediate results (such as a previously predicted sequence of tokens) are fed back.

According to some examples, the output of the encoder is input into the decoder.

Further, the transformer network may comprise a classification module or unit configured to map the output of the encoder or decoder to a set of learned outputs in the form of the image acquisition information.

In particular, the transformer network may be embodied as a vision transformer. The vision transformer may be configured to break down input k-space images into frequency patches and tokenize them (extracting representation vectors), before applying the tokens to a standard transformer architecture. The vision transformer may comprise an attention mechanism configured to repeatedly transform representation vectors of image patches for increasingly incorporating semantic relations between image patches in a k-space image.

According to some examples, the vision transformer may be obtained by training a masked autoencoder. A masked auto-encoder comprises two vision transformers put end-to-end. The first one takes in k-space image patches with positional encoding in frequency space, and outputs vectors representing each patch. The second one takes in vectors with frequency-positional encodings and outputs k-space image patches again. During training, both vision transformers are used. A k-space image is cut into patches. The second vision transformer takes the encoded vectors and outputs a reconstruction of the full k-space image. During use, the first vision transformer may be used as encoder and/or the second vision transformer may be used as generative Al function.

For a review on transformer networks, reference is made to Vaswani et al., “Attention Is All You Need”, in arXiv: 1706.03762, Jun. 12, 2017, the contents of which are herein included by reference in their entirety.

An advantage of transformer networks is that, due to the attention mechanism, transformer networks can efficiently deal with long-range dependencies in input data. Further, encoders used in transformer networks are capable of processing data in parallel which saves computing resources in inference. Moreover, decoders of transformer networks, due the auto-regression, are able to iteratively generate a sequence of output tokens with great confidence.

FocalNet is short for focal modulation network. A FocalNet is a neural network that uses a focusing mechanism to enable the model's interaction with the input, in this case the k-space image. Specifically, FocalNets use a lightweight element-wise multiplication as a focusing operator to see or interact with the input with the proposed modulator. The modulator is computed with a focal aggregation procedure in two steps: focal contextualization to extract contexts from local to global regions at different granularity levels and gated aggregation to condense all context features at different granularity levels into the modulator. For a review on FocalNets, reference is made to Yang et al., Focal Modulation Networks, arXiv:2203.11926, the contents of which are herein included by reference in their entirety.

The inventors have recognized that FocalNet may outperform attention-based neural networks when dealing with k-space images.

According to some examples, the medical image is a 3D medical image, and the method further comprises obtaining an image slice of the medical image, wherein the k-space image is generated by transforming the image slice into the frequency domain.

Thereby, obtaining the image slice may comprise selecting the image slice from a plurality of slices comprised in the medical image or defining the image slice in the 3D medical image.

According to some examples, obtaining the image slice may comprise determining a suitability measure (or quality score) of a plurality of candidate slices of the medical image, wherein the suitability measure indicates a suitability of the slice for the ensuing steps. According to some examples the suitability measure may be based on a position of the slice in the medical image (wherein marginal slices may have a lower suitability than more central slices) and/or an image content of the slice (wherein image slices with imaging artifacts may have a lower suitability).

According to some examples, the suitability measure may be determined by a further trained function which has been configured to obtain the image slice. The further trained function may be based on a convolutional neural network. Further, the further trained function may be part of the trained function. The further trained function may be trained by processing a plurality of slices from the medical image with the trained function and comparing the result to a ground truth for the image acquisition information. The result of the comparison may be fed back to the further trained function for optimizing the slice obtaining process.

By obtaining the image slice, information can be pre-selected from the medical image which optimally represents the medical image. This may increase the quality of the processing results and the efficiency of the method.

According to an aspect, the image acquisition information comprises an image acquisition parameter and/or an information about a body part depicted in the medical image.

An image acquisition parameter may comprise the type and/or make of the imaging modality used and/or control parameter settings of the imaging modality used during the acquisition of the medical image. To provide an example, image acquisition parameters may comprise the following information: type Chest-CT scan, bolus agent: xyz, modality: Siemens Healthineers CT scanner, model number: 12345, kilovoltage peak: xxx, milliampere seconds: yyy. The information about the body part may comprise an indication of the body part depicted and/or the body compartments therein comprised, and/or an indication of findings comprised in the body part/compartments. To provide an example, information about the body part may comprise: chest area, showing the lung, rib cage, spine of the patient with a lung nodule in the upper left lung lobe.

With the image acquisition parameters and/or information about body parts valuable information is provided which determines the further processing steps in the further diagnostic image processing workflow. With that, these steps can be more readily triggered automatically without user input.

According to an aspect, the medical image has been acquired with a magnetic resonance acquisition procedure using a magnetic resonance medical imaging modality, and the image acquisition parameter relates to the image weighting and/or the magnetic resonance sequence used in the acquisition procedure. In other words, determining the image acquisition parameter comprises classifying the medical image according to the image weighting and/or the magnetic resonance sequence used.

The image weighting may indicate on which relaxation effect the magnetic resonance imaging was focused on. Each tissue returns to its equilibrium state after excitation by independent relaxation processes of T1 (spin-lattice; that is, magnetization in the same direction as the static magnetic field) and T2 (spin-spin; transverse to the static magnetic field). To create a T1-weighted image, magnetization is allowed to recover before measuring the MR signal by changing the repetition time. This image weighting is useful for assessing the cerebral cortex, identifying fatty tissue, characterizing focal liver lesions, and in general, obtaining morphological information, as well as for post-contrast imaging. To create a T2-weighted image, magnetization is allowed to decay before measuring the MR signal by changing the echo time. This image weighting is useful for detecting edema and inflammation, revealing lesions and abnormalities.

Within the T1/T2 weightings there may be more subtle variations. The T2* weighting builds on a distribution of resonance frequencies around the ideal. Over time, this distribution can lead to a dispersion of the distribution of magnetic spin vectors. This results in dephasing. For molecules that are not moving, the deviation from ideal relaxation is consistent over time, and the signal can be recovered by performing a spin echo experiment. T2*-weighted sequences are used to detect deoxygenated hemoglobin, methemoglobin, or hemosiderin in lesions and tissues. Diseases with such patterns include intracranial hemorrhage, arteriovenous malformation, cavernoma, hemorrhage in a tumor, punctate hemorrhages in diffuse axonal injury, superficial siderosis, thrombosed aneurysm, phleboliths in vascular lesions, and some forms of calcification.

The magnetic resonance sequence may relate to the succession of pulse sequences and pulsed field gradients a specimen is subjected to. By varying the parameters of the pulse sequence, different contrasts may be generated between tissues based on the relaxation properties. In other words, different image weightings may be generated. Moreover, different weightings are possible for different sequences. For instance, BLADE (see below) may be combined with a T1, T2, or STIR weighting.

The inventors have recognized that the step of transforming real medical images from magnetic resonance sources provides valuable insights into the image acquisition parameters. This is because in magnetic resonance imaging, complex values are sampled in k-space during the measurement in a premeditated scheme controlled by a pulse sequence, i.e., an accurately timed sequence of radiofrequency and gradient pulses. During acquisition, the k-space often refers to the temporary image space, usually a matrix, in which data from digitized magnetic resonance signals are stored during data acquisition. At the end of the scan, the data are mathematically processed to produce a final medical image. Thus, the matrix holds raw data before reconstruction. At the stage of reading the medical image, the raw data is most often no longer available as typically only the reconstructed image is stored (e.g., in a PACS). Accordingly, the proposed method step of transforming the medical image (back) into the k-space may reveal or put the focus on information which more directly relates to the image acquisition process. This may lead to better predictions of the image acquisition information and allow for more readily automating of the reading and reporting workflow.

Specifically, the magnetic resonance sequence and/or the image weighting may provide important cues on how the medical image should to be processed for image reading and what kind of CAD-tools are to be applied on the medical image. This is because a T2* weighted image or a corresponding sequence may indicate a very different underlying clinical question and suggest very different subsequent processing steps as a T1 weighted image.

Of note, the transformation into the k-space is also advantageous with other image acquisition techniques such as computed tomography, as the method also in those cases may open up an additional layer of information.

According to some examples, the image weighting at least comprises or distinguishes between a T1, T2, T2*, proton density (PD), steady-state free precession (SSFP), Susceptibility-weighted (SWI), Short tau inversion recovery (STIR), Inversion recovery (IR), Double inversion recovery (DIR), Diffusion (DWI), Perfusion (PWI), susceptibility-weighted imaging (SWI), Blood-oxygen-level dependent (BOLD), and/or Time-of-flight (ToF) weighting.

According to some examples, the imaging sequence at least comprises or distinguishes between a spin-echo-sequence, a gradient-echo sequence, an inversion recovery sequence, a MR angiography sequence, a saturation recovery sequence, an echo-planar sequence, a spiral pulse sequence, an in-and out-of-phase imaging sequence, and sub-sequences and combinations of the aforesaid.

According to some examples, the imaging sequence distinguishes between at least two different spin-echo-sequences. In other words, determining the image acquisition information comprises classifying the medical image according to at least two different spin-echo-sequences.

According to other examples, the imaging sequence distinguishes between BLADE and HASTE.

BLADE is a proprietary sequence of the Siemens Healthineers AG which reduces the sensitivity to movement in magnetic resonance scanning. It is a technique that incorporates a k-space trajectory radial in nature, reduces motion artifacts and helps visualizing the smallest lesions.

HASTE is a spin-echo sequence trademarked by Siemens Healthineers AG. It is a single-shot technique. This means that data from each line of the k-space is obtained after a single 90°-excitation pulse.

Distinguishing between spin-echo-sequences and/or between such as BLADE and HASTE is important for the subsequent processing steps and, in particular, for selecting the right hanging protocol. At the same time, the differences between real image data of different spin-echo sequences are subtle making this process difficult. The same holds true for distinguishing between BLADE and HASTE. In this regard, the inventors have recognized that such differences can be more readily resolved in k-space.

According to an aspect, the trained function is additionally applied on the medical image in the step of determining.

In other words, not only the k-space image data but also the real image data is input in the trained function. In turn, the trained function may also have been trained on the real image data to base the prediction of the image acquisition information on this data also.

This has the advantage that properties which are more readily derivable from the real image such as the body parts and compartments shown can be more readily inferred from the data. Moreover, this enables to use already available classifiers configured to be applied on real image data for setting up the trained function. This may include segmentation algorithms configured to recognize and segment organs of a patient, or detection and/or classification algorithms configured to detect and classify image patterns such as lesions or other abnormalities.

According to an aspect, the method further comprises generating a merged image based on the medical image and the k-space image, wherein, in the step of determining, the trained function is applied on the merged image.

According to some examples, the k-space image has the same size and/or the same dimensions as the medical image. In particular, it may have as many individual values as the medical image has pixels/voxels. Moreover, there may be a k-space value for each pixel/voxel of the medical image. According to some examples, the k-space values may be overlayed over or added to the pixel-or voxel values of the medical image. According to other examples, the k-space values may be added to the medical image in the form of an additional layer or dimension such that each pixel or voxel of the medical image has an additional dimension provided by the corresponding k-space value. According to some examples, the k-space image data is added as additional image channel(s) to the medical image.

Generating the concatenated image may have the advantage of an intrinsic registration which may facilitate the identification of relations between the image space and the k-space. In turn, this may lead to more meaningful predictions and better results in the ensuing workflow automation.

According to an aspect, a computer-implemented method for displaying a representation of a medical image is provided. The method comprises a plurality of steps. One step is directed to receiving the medical image from a database. Another step is directed to determining an image acquisition information of the medical image according to any one of the aspects and examples herein described. Another step is directed to generating a representation of the medical image for displaying in a user interface based on the image acquisition information. Another step is directed to displaying the representation in the user interface.

The representation may be generated by processing the medical image wherein the processing depends on the image acquisition parameters.

The representation may comprise one or more two-dimensional representation images rendered from the medical image. The representation images may comprise a plurality of image pixels. In particular, the representation images may be two-dimensional renderings of the medical image or of different views of the medical image. Two-dimensional renderings may, in general, rely on known rendering procedures, including ray-casting, ray-tracing, texture-rendering or the like. Thereby, the views and the rendering may depend on the image acquisition information. For instance, the image acquisition information may suggest a certain rendering or pre-processing and/or a particular view.

Further, the representation may comprise a graphical user interface in which the representation images are included at a predefined position. In particular, the graphical user interface may be specifically configured to derive a certain medical diagnosis based on the medical image. Via the graphical user interface, the user may inspect the medical image, make measurements and record a medical diagnosis (e.g., in the form of a medical report).

By automatically generating the representation based on the image acquisition parameters, views which will likely be required based on the image acquisition information are automatically generated and offered to a user. This relieves the user from the routine but tedious task of setting up the representation herself for the further diagnosis of the medical image.

According to an aspect, the step of generating comprises determining a displaying setting based on the image acquisition information and applying the selected displaying setting for generating the representation, the displaying setting being selected from: a contrast setting, a brightness, an intensity windowing, an image enhancement, a look-up table, a viewing plane, a segmentation mask, a zoom level or panning, and/or a volumetric rendering parameter.

By automatically, determining displaying settings, the user is automatically provided with appropriate parameters and does not have to set these herself.

According to an aspect, the representation comprises a volumetric rendering of the medical image, in particular, generated with a path-tracing-or ray-casting-based rendering process, and the displaying setting comprises a volumetric rendering parameter for generating the volumetric rendering.

In ray casting, simulated rays emanating from the eye of an imaginary observer are transmitted through the examined body or the examined object (cf. Levoy: “Display of Surfaces from Volume Data”, IEEE Computer Graphics and Applications, issue 8, no. 3, May 1988, pages 29-37). Along the rays, RGBA values are determined for sampling points from the voxels and combined to form pixels for a two-dimensional image via alpha compositing or alpha blending. Here, the letters R, G and B in the expression RGBA represent the color components red, green and blue, from which the color contribution of the corresponding sampling point is composed. A represents the ALPHA value, which represents a measure for the transparency at the sampling point. The respective transparency is used in the superposition of RGB values at sampling points to form the pixel. Lighting effects are usually taken into account via a lighting model within the scope of a method referred to as “shading”.

A further method for volume rendering is the so-called path tracing method (cf. Kajiya: “The rendering equation”, ACM SIGGRAPH Computer Graphics, issue 20, no. 4, August 1986, pages 143-150). Here, a plurality of simulated rays is shot into the volume data per visualization pixel, said simulated rays then interacting with the volume, i.e., are reflected, refracted or absorbed, wherein at least one random ray is generated every time (except in the case of absorption). Each simulated ray thus finds its path through the volume data. The more virtual rays are used per visualization pixel, the better the image. Here, use can be made, in particular, of the processes and methods described in EP 3 178 068 B1. The contents of EP 3 178 068 B1 are incorporated herein in full by reference.

Accordingly, the displaying settings may specify parameters for the path-and/or ray-casting process such as zoom levels, viewing angles, transfer functions, texture values, number of rays, transparency levels, scene illuminations and so forth.

On the one hand, such methods allow particularly realistic visualizations to be generated. This provides the human recipient with an instructive picture of the imaging examination and its outcome. On the other hand, since the volumetric image rendering is triggered automatically, the user does not need to get involved which spares the user of familiarizing herself with the subtleties of a volumetric rendering pipeline (which may be complex).

According to an aspect, the method further comprises selecting, based on the image acquisition information, a hanging protocol including a rule set for displaying one or more representations of a medical image in a user interface, wherein, in the step of displaying, the representation is displayed based on the hanging protocol.

Current reading and reporting systems use general techniques known as “hanging protocols” to format the display or layout of medical images or excerpts from medical images. Hanging protocols allow a user to specifically set displaying environments according to modality, anatomy, and procedure. Hanging protocols present one or more perspectives or views (e.g., in the form of the aforementioned representations) of the medical image to a user such as a radiologist. Representations may be grouped and located in a graphical user interface. In addition, hanging protocols may comprise rules or instructions for obtaining additional information such as comparative medical images acquired before or after the medical image or for applying certain image analysis tools.

According to some examples, the hanging protocol may be selected from a plurality of predefined hanging protocols. The predefined hanging protocols may be configured according to different use cases and the step of selecting may comprise identifying a use case based on the image acquisition information and selecting the hanging protocol corresponding to the identified use case.

By selecting the appropriate hanging protocol, the user is automatically provided with a user interface specifically adapted for the image data and the diagnostic task. This not only relieves the user but also automates the image processing towards the provision of a medical diagnosis.

According to an aspect, the method further comprises retrieving, from the database and based on the image acquisition information, a comparative medical image, processing the comparative medical image so as to generate a comparative representation for displaying in the user interface, and displaying the comparative representation in the user interface.

According to some examples, the comparative medical image may have the same or similar image acquisition information as the medical image. According to some examples, the comparative medical image may be processed in the same way as the medical image. For instance, the comparative medical images may be based on the same magnetic resonance sequence and/or image weighting as the medical image. With that, the comparative medical image can be more readily compared to the medical image and/or the same measurements may be made.

The comparative medical image may relate to the same patient as the medical image. The comparative medical image may relate to a different patient as the medical image. In particular, the comparative medical image may have a degree of similarity to the medical image. The comparative medical image may be obtained from a database of comparative medical images. Further, the comparative medical image may be obtained from an electronic medical textbook. The comparative medical image may be associated with a verified medical diagnosis.

By offering a comparative medical image to a user, the user can be provided with additional information for deriving a medical diagnosis.

According to some examples, the medical image was acquired from the patient at a first point in time and the comparative medical image was acquired from the patient at a second point in time different than the first point in time.

In other words, a prior or subsequent medical image of the patient may be automatically retrieved which has comparable image acquisition information. In particular, this may mean that the medical image shows a body part of the patient at the first point in time and the comparative medical image shows the body part at a second point in time. With that, the user can more easily determine a development of the health condition of the patient and, thus, may come up with a better diagnosis. In this regard, since the comparative medical image is automatically retrieved based on the image acquisition information, the user is spared from the time-consuming task of having to search the database for appropriate priors, for instance.

According to an aspect, the method further comprises selecting, based on the image acquisition information, an image processing tool configured to provide an image processing result, apply the selected image processing tool so as to generate the image processing result, and displaying the image processing result in the user interface.

According to some examples, the image processing tools may also be applied to any comparative medical images.

The image processing tool may be selected from a plurality of available image processing tools. The image processing tools may be specific to certain use cases/image acquisition information. For example, the plurality of image processing tools may comprise tools specific for a certain modality, anatomy and/or procedure. Specifically, there might be specific image processing tools for certain magnetic resonance sequences.

Further, the image processing tool may be selected from one or more segmentation tools, one or more detection/classification tools, one or more change detection tools etc. Accordingly, the image detection result may comprise: a detection result of a medical finding in the medical image, a classification of a medical finding in the medical image, a segmentation of the medical image, and/or a change detected in the medical image.

By automatically identifying and applying the image processing tool, results can be automatically generated. The user is provided with cues for arriving at a medical diagnosis without having to search the library of available tools for appropriate ones.

According to some examples, the method further comprises selecting, based on the image acquisition information, a reporting template for producing a medical report corresponding to the medica image, and providing the reporting template via the user interface.

A reporting template may be a pre-configured data structure or building block or module on the basis of which a structured medical report may be generated. A medical report may be generated based on at least one reporting template.

Selecting the reporting template may comprise a selection from a plurality of reporting templates. Each reporting template may be specific to a certain image acquisition information. For instance, a certain reporting template may be associated to a magnetic resonance scan of the brain, while another reporting template is associated to a chest CT scan.

Each reporting template may specify one or more data fields which have to be addressed or filled for completing the medical report. Further, a reporting template may comprise one or more pull-down menus with items a user can select. As such, a reporting template may also be conceived as an input form or mask structuring the information to be provided for a given diagnostic task.

According to some examples, the method may further comprise pre-filling the reporting template based on the image acquisition information and/or any other image processing results, e.g., as obtained by applying image processing tools.

By fetching appropriate reporting templates, the user is automatically provided with appropriate template data structures. In turn, the user is relieved from the burden of having to search for correct template data structure on her own in potentially vast databases.

According to an aspect, the method further comprises determining, based on the image acquisition information, a designated recipient of the medical image in a medical information system comprising a plurality of possible recipients, the possible recipients comprising one or more of a database, an image processing server, a worklist of a user, a reading and reporting workplace of a user, and/or a data exchange server for exchanging data to the outside of the healthcare information system. Optionally, the method may include forwarding/transmitting the medical image to the designated recipient.

By identifying designated recipients, the medical image may be automatically routed to the correct place in the healthcare information system. If, for instance, the image acquisition information indicates that a brain image was taken with a certain MR-pulse sequence indicating a certain clinical question the image may be automatically routed to a clinical expert for that kind of studies.

According to an aspect, a computer-implemented method for providing a trained function for deriving an image acquisition information form a medical image is provided. The method comprises a plurality of steps. A first step is directed to provide a training data set comprising a medical image and a verified image acquisition information. A further step is directed to transforming the medical image (or data of the medical image) into the frequency domain so as to obtain a k-space image. A further step is directed to apply the trained function to the k-space image so as to obtain an intermediate image acquisition information. A further step is directed to compare the intermediate image acquisition information with the verified image acquisition information. A further step is directed to adapt the trained function based on the step of comparing. Another step is directed to provide the adapted trained function.

According to a further aspect, a computer-implemented method for providing a trained function for deriving an image acquisition information form a medical image which has been acquired with a magnetic resonance acquisition procedure is provided. The method comprises a plurality of steps. A first step is directed to provide a training data set comprising a raw magnetic resonance image in the frequency domain and a verified image acquisition information, wherein the medical image is to be reconstructed from the raw image. A further step is directed to apply the trained function to the raw image so as to obtain an intermediate image acquisition information. A further step is directed to compare the intermediate image acquisition information with the verified image acquisition information. A further step is directed to adapt the trained function based on the step of comparing. Another step is directed to provide the adapted trained function.

The latter aspect has the advantage that the training data can be directly obtained from the magnetic resonance machine without requiring image reconstruction or transformation steps.

According to an aspect, a system for providing an image acquisition information of a medical image is provided. The system comprises an interface unit and a computing unit. The computing unit is configured to receive the medical image via the interface unit, to transform the medical image into the frequency domain so as to obtain a k-space image, to determine the image acquisition information by applying a trained function to the k-space image, and to provide the image acquisition information via the interface unit.

According to a further aspect, a system for providing control signals for displaying a representation of a medical image is provided. The system comprises an interface unit and a computing unit. The computing unit is configured to receive the medical image from a database via the interface unit, to transform the medical image into the frequency domain so as to obtain a k-space image, to determine the image acquisition information by applying a trained function to the k-space image, to generate control signals for controlling a user interface to display a representation of the medical image, the representation being generated according to (based on) the image acquisition information, and to provide the control signals to the user interface via the interface unit.

The computing unit(s) may be realized as a data processing system or as a part of a data processing system. Such a data processing system can, for example, comprise a cloud-computing system, a computer network, a computer, a tablet computer, a smartphone and/or the like. The computing unit can comprise hardware and/or software. The hardware can comprise, for example, one or more processors, one or more memories and combinations thereof. The one or more memories may store instructions for carrying out the method steps according to one or more example embodiments. The hardware can be configurable by the software and/or be operable by the software. Generally, all units, sub-units, or modules may at least temporarily be in data exchange with each other, e.g., via a network connection or respective interfaces. Consequently, individual units may be located apart from each other.

The interface unit may comprise an interface for data exchange with a local server or a central web server via internet connection for receiving the medial images.

The user interface may be adapted to interface with one or more users of the system, e.g., by displaying the result of the processing of the computing unit to the user (e.g., in a graphical user interface) or by allowing the user to make inputs for arriving at a medical diagnosis.

According to other aspects, one or more example embodiments further relates to an integrated data management system (or healthcare information system) comprising the above system and an image archiving system configured to acquire, store and/or forward medical images. Thereby, the interface unit may be configured to receive the medical image data set form the image archiving system. According to some examples, the image archiving system may be realized as a cloud storage or as a local or spread storage, e.g., as a PACS (Picture Archiving and Communication System).

According to other aspects, the systems are adapted to implement the inventive method in their various aspects for providing a candidate medical finding. The advantages described in connection with the method aspects may also be realized by the correspondingly configured systems' components.

According to another aspect, the present invention is directed to a computer program product comprising program elements which induce a computing unit of systems herein described to perform the steps according to one or more of the above method aspects and examples as herein described, when the program elements are loaded into a memory of the computing unit.

According to another aspect, the present invention is directed to a computer-readable medium on which program elements are stored that are readable and executable by a computing unit of systems herein described to perform the steps according to one or more method aspects and examples as herein described, when the program elements are executed by the computing unit.

The realization of example embodiments by a computer program product and/or a computer-readable medium has the advantage that already existing providing systems can be easily adapted by software updates in order to work as proposed by one or more example embodiments.

The computer program product can be, for example, a computer program or comprise another element next to the computer program as such. This other element can be hardware, e.g., a memory device, on which the computer program is stored, a hardware key for using the computer program and the like, and/or software, e.g., a documentation or a software key for using the computer program. The computer program product may further comprise development material, a runtime system and/or databases or libraries. The computer program product may be distributed among several computer instances.

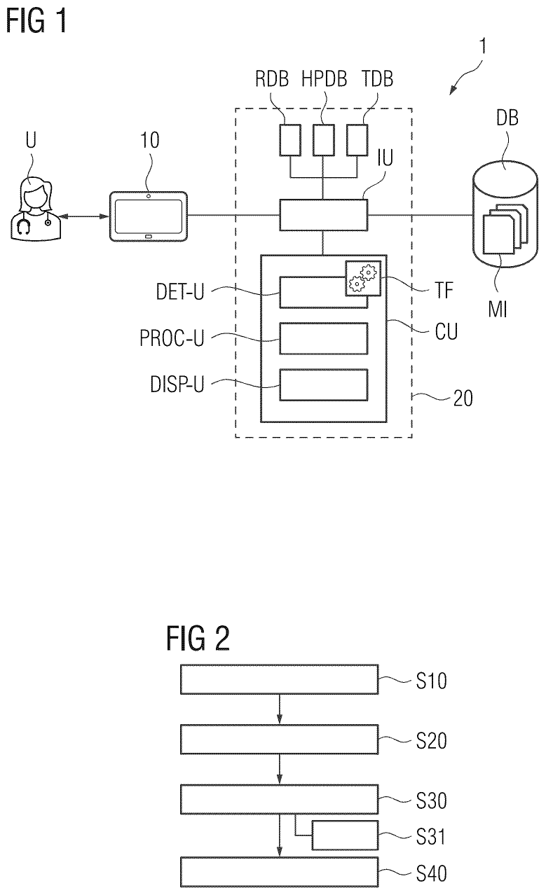

FIG. 1 depicts a healthcare information system 1 for providing an image acquisition information IAI and/or a representation RE of a medical image MI. In this regard, healthcare information system 1 is adapted to perform the methods according to one or more embodiments, e.g., as further described with reference to FIGS. 2 to 6.

A user U of healthcare information system 1, according to some examples, may generally relate to a healthcare professional such as a physician, clinician, technician, radiologist and so forth.

Healthcare information system 1 may comprises a user interface 10 and a processing system 20. Further, system 1 may comprise or be connected to a database DB generally configured for storing and/or forwarding medical images MI and supplementary (non-image) information. The components of the healthcare information system 1 may also be referred to as “recipients” as they may receive data such as medical images MI and information derived therefrom.

The database DB may comprise one or more storage devices for medical images MI which may be realized in the form of one or more cloud storages, local or spread storage modules, e.g., as a PACS (Picture Archiving and Communication System).

According to some examples, the healthcare information system may comprise one or more medical imaging modalities (not shown) for acquiring medical images MI, such as a computed tomography system, a magnetic resonance system, an angiography (or C-arm X-ray) system, a positron-emission tomography system, a mammography system, an X-ray system, or the like.

Medical images MI may be three-dimensional image data sets acquired, for instance, using an X-ray system, a computed tomography system or a magnetic resonance imaging system or other systems. The image information may be encoded in a three-dimensional array of m times n times p voxels. Medical images MI may include a plurality of image slices which are stacked in a stacking direction to span the image volume covered by the medical image MI.

Further, medical images MI may comprise two-dimensional medical image data with the image information being encoded in an array of m times n pixels. According to some examples, these two-dimensional medical images may have been extracted from three-dimensional medical image MI.

An ensemble of voxels or pixels may be designated as image data of the respective medical image MI in the following. In general, any kind of imaging modalities and scanners may be used for acquiring such image data. Generally, medical images MI may show a body part or an anatomical region or an anatomic object of a patient which may comprise various anatomies and organs. Considering the chest area as a body part, medical images MI might, for instance, depict the lung lobes, the rib cage, the heart, lymph nodes, and so forth.

Medical images MI may be formatted according to the DICOM format. DICOM (=Digital Imaging and Communications in Medicine) is an open standard for the communication and management of medical imaging information and related data in healthcare informatics. DICOM may be used for storing and transmitting medical images and associated information enabling the integration of medical imaging. A DICOM data object consists of a number of attributes, including items such as the patient's name, ID, etc., and also special attributes containing the image pixel data and metadata extracted from the image data. The metadata may be stored in the so-called DICOM header.

User interface 10 may comprise a display unit and an input unit. User interface 10 may be embodied by a mobile device such as a smartphone or tablet computer. Further, user interface 10 may be embodied as a workstation in the form of a desktop PC or laptop. The input unit may be integrated in the display unit, e.g., in the form of a touch screen. As an alternative or in addition to that, the input unit may comprise a keyboard, a mouse or a digital pen and any combination thereof. The display unit may be configured for displaying representations RE of the medical image MI, medical report templates RT, image processing results FI, in a graphical user interface GUI, wherein all elements to be shown may be arranged according to a hanging protocol HP.

User interface 10 may further comprise an interface computing unit configured to execute at least one software component for serving the display unit and the input unit in order to provide a graphical user interface for allowing the user to select a target patient's case to be reviewed and making various inputs. In addition, the interface computing unit may be configured to communicate with the database DB or processing system 20 for receiving the medical images MI and any supplementary information. The user U may activate the software component via user interface 10 and may acquire the software component, e.g., by downloading it from an internet application store. According to an example, the software component may also be a client-server computer program in the form of a web application running in a web browser. The interface computing unit may be a general processor, central processing unit, control processor, graphics processing unit, digital signal processor, three-dimensional rendering processor, image processor, application specific integrated circuit, field programmable gate array, digital circuit, analog circuit, combinations thereof, or other now known devices for processing image data. User interface 10 may also be embodied as a client.

The processing system 20 may comprise a computing unit CU and an interface unit IU. Further, the processing system 20 may comprise or be connected to a plurality of dedicated repositories or databases including a reporting database RDB, a tool database TDB, and a hanging protocol database HPDB. According to some examples, the databases RDB, TDB, HPDB may be part of the healthcare information system 1.

The reporting database RDB is a storage device such a cloud or local storage serving as an archive for preconfigured reporting templates RT.

Thereby, a reporting template RT may be seen as a building block for a medical report. Reporting template RT may be configured for editing by the user via user interface 10. Reporting template RT may comprise one or more data fields into which diagnostic information specific for the patient and/or the underlying medical image MI may be specified. The data fields may be empty fields or placeholders for various kinds of data such as text, measurement values or images.

A reporting template RT may be specific to a certain diagnostic use case (which may be indicated by the image acquisition information as herein described).

The hanging protocol database HPDB is a storage device such a cloud or local storage serving as an archive for preconfigured hanging protocols HP.

A hanging protocol HP may comprise a series of rules in the form of computer-executable instructions for optimally arranging medical information, in particular, medical images in a graphical user interface according to a dedicated use case. A hanging protocol may set out which kind of representations of a medical image MI and other information are to be produced and where these elements are to be shown in the graphical user interface GUI.

The tool database TDB is a storage device such as a cloud or a local storage serving as a repository for preconfigured image processing tools IPT.

Image processing tools IPT are generally configured to be applied to medical image data MI. In other words, these are tools which are configured to process image data in order to provide a corresponding processing result FI. The image processing result may be related to a medical finding FI. According to some examples, the processing tools IPT may be specialized for a certain use-case such as a type of medical image data (e.g., MR image data or CT image data) and/or a certain type of image processing result. For instance, one of the image processing tools IPT may be configured to detect lesions in an MR scan of a patient which was acquired with a HASTE sequence, while another image processing tool IPT may be configured to segment bones in a bone window of a CT scan. Generally, the tool database IDB may comprise all image processing algorithms which are available for processing all kinds of image data which may occur at a certain healthcare facility or diagnostic workplace.

Computing unit CU may be a processor. The processor may be a general processor, central processing unit, control processor, graphics processing unit, digital signal processor, three-dimensional rendering processor, image processor, application specific integrated circuit, field programmable gate array, digital circuit, analog circuit, combinations thereof, or other now known device for processing image data. The processor may be single device or multiple devices operating in serial, parallel, or separately. The processor may be a main processor of a computer, such as a laptop or desktop computer, or may be a processor for handling some tasks in a larger system, such as in the medical information system or the server. The processor is configured by instructions, design, hardware, and/or software to perform the steps discussed herein. Further, processing system 20 may comprise a memory such as a RAM for temporally loading the medical images MI and any intermediate processing results. According to some examples, such memory may as well be comprised in user interface 10.

Processing system 20 may comprise sub-units DET-U, PROC-U, DISP-U configured to process the medical image MI and in order to provide an image acquisition information IAI and/or further process the medical image MI based on the image acquisition information IAI.

Sub-unit DET-U is configured to determine image acquisition information IAI for medical images MI. The image acquisition information IAI comprises the framework under which the medical image MI was acquired. This includes the type of modality and imaging parameters used. Sub-unit DET-U is specifically configured to extract the image acquisition information IAI directly from the image data of the medical image MI, that is, the pixels and voxels. Non-image data is only considered as an auxiliary source. To do so, sub-unit DET-U is configured to transform the medical image MI into frequency domain (e.g., by relying on a Fourier transformation) so as to generate a k-space image KI and derive the image acquisition information IAI from the k-space image KI. Specifically, sub-unit DET-U is configured to run an accordingly configured trained function TF which has been trained to derive the image modality used and imaging parameters etc. from k-space images KI.

Sub-unit PROC-U is configured to leverage the image acquisition information IAI for further processing the medical image MI for deriving a medical diagnosis from the medical image MI by a user U. This may involve providing an image processing result by selecting an image processing tool IPT from the tool database TDB according to the image acquisition information IAI and applying it to the medical image MI. Further, sub-unit PROC-U may be configured to generate one or more representations RE from the medical image MI for displaying to the user U which fit the image acquisition information IAI. Further, sub-unit PROC-U may use the image acquisition information IAI to select suitable reporting templates RT and hanging protocols HP from the reporting database RDB or the hanging protocol database HPDB.

Sub-unit DISP-U is a displaying module or unit. Specifically, sub-unit DISP-U may be configured to use the hanging protocol HP selected by the processing unit PROC-U and arrange any representations RE, image processing results FI, reporting templates RT, or any other data elements in a graphic user interface GUI according to the hanging protocol HP.

The designation of the distinct sub-units DET-U, PROC-U, DISP-U is to be construed by way of example and not as a limitation. Accordingly, sub-units DET-U, PROC-U, DISP-U may be integrated to form one single unit (e.g., in the form of “the computing unit”) or can be embodied by computer code segments configured to execute the corresponding method steps running on a processor or the like of processing system 20. The same holds true with respect to the interface computing unit. Each sub-unit DET-U, PROC-U, DISP-U and the interface computing unit may be individually connected to other sub-units and/or other components of the system 1 where data exchange is used to perform the method steps.

Processing system 20 and the interface computing unit(s) together may constitute the computing unit of the system 1. Of note, the layout of this computing unit, i.e., the physical distribution of the interface computing unit and sub-units DET-U, PROC-U, DISP-U is, in principle, arbitrary. Specifically, processing system 20 may also be integrated in user interface 10. As already mentioned, processing system 20 may alternatively be embodied as a server system, e.g., a cloud server, or a local server, e.g., located on a hospital or radiology site. According to such implementation, user interface 10 could be designated as a “frontend” or “client” facing the user, while processing system 20 could then be conceived as a “backend” or server. Communication between user interface 10 and processing system 20 may be carried out using the https-protocol, for instance. The computational power of the system may be distributed between the server and the client (i.e., user interface 10). In a “thin client” system, the majority of the computational capabilities exists at the server. In a “thick client” system, more of the computational capabilities, and possibly data, exist on the client.

Individual components of system 1 may be at least temporarily connected to each other for data transfer and/or exchange. User interface 10 communicates with processing system 20 via interface unit IU to exchange, e.g., medical images MI, elements of a graphical user interface GUI or any user input made. Further, processing system 20 may communicate interface unit IU with the database 40 and/or the dedicated database TDB, HPDB, RDB. The interface unit IU may be realized as hardware-or software-interface, e.g., a PCI-bus, USB or fire-wire. Data transfer may be realized using a network connection. The network may be realized as local area network (LAN), e.g., an intranet or a wide area network (WAN). Network connection is preferably wireless, e.g., as wireless LAN (WLAN or Wi-Fi). Further, the network may comprise a combination of different network examples.

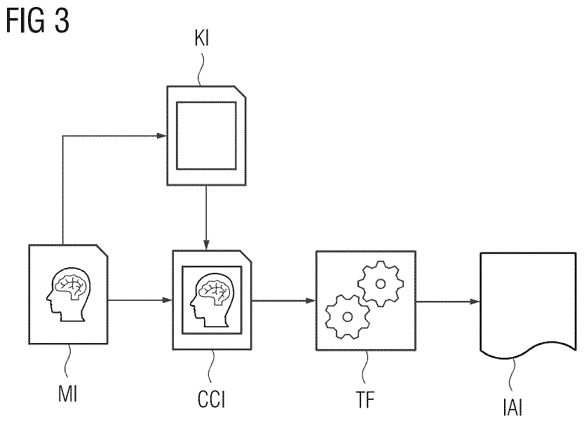

FIG. 2 depicts a method for providing an image acquisition information IAI according to an embodiment. Corresponding data streams are illustrated in FIG. 3. The method comprises several steps. The order of the steps does not necessarily correspond to the numbering of the steps but may also vary between different embodiments. Further, individual steps or a sequence of steps may be repeated.

At step S10, a medical image MI of a patient is obtained. This may involve selecting the medical image MI from a plurality of cases, e.g., stored in the database DB. The selection may be performed manually by the user U, e.g., by selecting appropriate image data in a graphical user interface GUI running in the user interface 10. Alternatively, the medical image MI may be provided to the computing unit CU by the user U by way of uploading the medical image MI to the computing unit CU. According to an example, the medical image MI has been acquired using a magnetic resonance imaging modality using a particular image weighting and magnetic resonance sequence.

At step S20, the medical image MI is transformed into the frequency domain so as to obtain a k-space image KI of the medical image MI. This may involve subjecting the medical image MI to a Fourier transform. To this and, a fast Fourier transform (FFT) algorithm may be applied to the medical image MI. The FFT-algorithm may be hosted at the computing unit CU.

Step S20 may comprise transforming the entire medical image MI into the frequency domain. According to other examples, step S20 may comprise selecting a representative part of the medical image MI, in particular, an image slice, and transforming the representative part only.

At step S30, an image acquisition information IAI is determined based on the k-space image KI and, optionally, additionally based on the medical image MI. This comprises inputting the data of the k-space image KI and, optionally, the data of the medical image MI in the trained function TF hosted at the computing unit CU.

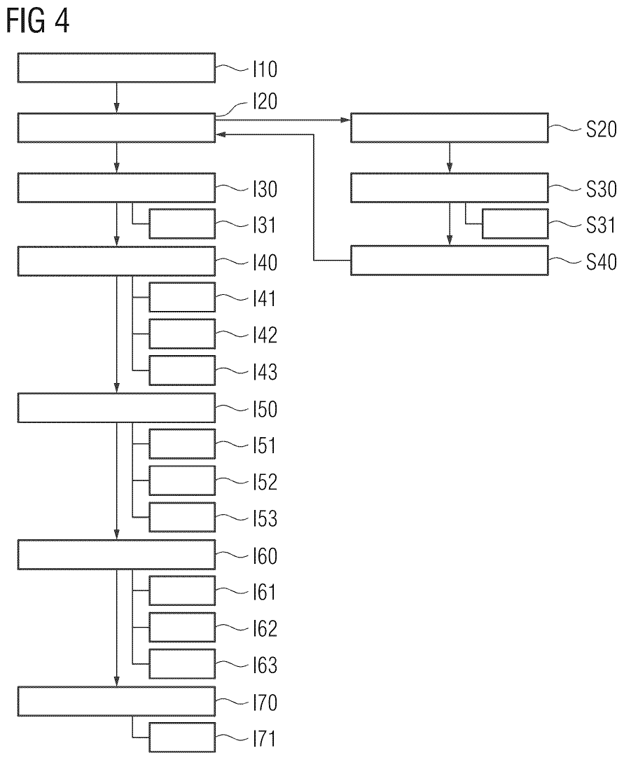

At optional sub-step S31, the data of the k-space image KI may be aligned with the image data of the medical image MI. As shown in FIG. 4, this may involve generating a concatenated image CCI. In the concatenated image CCI, the data of the k-space image KI may be overlayed over the image data of the medical image MI. As an alternative, the data of the k-space image may be added as a further image channel in the medical image MI, e.g., alongside the pixel-or voxel-vise intensity values. Due to the alignment of sub-step S31 every pixel/voxel of the medical image Mi may be assigned a k-space value. The concatenated image CCI may then be input in the trained function TF. That way, the trained function TF is provided with both the k-space and the real image data and, furthermore, with a relation between both.

At step S40, the image acquisition information IAI is provided. This may involve showing the image acquisition information IAI in the user interface 10, e.g., in a suitable graphical user interface GUI. Moreover, step S40 may comprise providing the image acquisition information IAI for subsequent image processing steps as shown in connection with FIGS. 4 to 6.

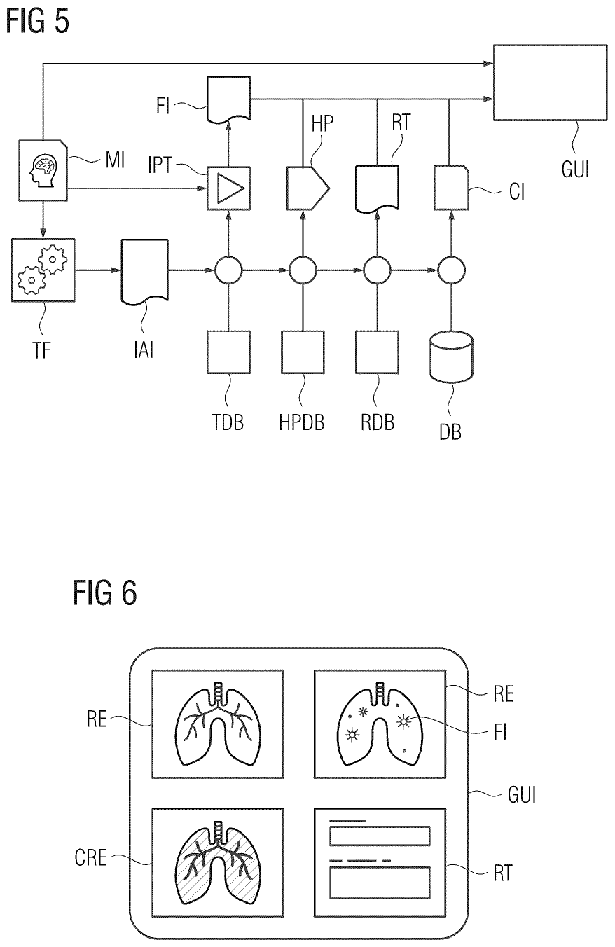

FIG. 4 depicts a method for displaying a representation RE of a medical image MI according to an embodiment. Corresponding data streams are illustrated in FIG. 5. FIG. 6 shows a corresponding graphical user interface GUI for displaying the representation RE and further information in a reading and reporting workflow. The method comprises several steps. The order of the steps does not necessarily correspond to the numbering of the steps but may also vary between different embodiments of the present invention. Further, individual steps or a sequence of steps may be repeated.

At step I10, the medical image MI is received. Thereby, step I10 substantially corresponds to step S10.

At step I20, the image acquisition information IAI is determined. As shown in FIG. 4, this may involve executing steps S20 to S40 as explained in connection with FIGS. 2 and 3. Optionally, step I20 may further comprise classifying the image acquisition information according to a plurality of predefined diagnostic use cases. According to some examples, the diagnostic uses cases may relate to a diagnostic task a user has to perform.

At step I30, the image acquisition information IAI is used to generate one or more appropriate representations RE of the medical image MI for displaying to a user U in the user interface 10. This may involve determining the type of representations RE coming into question for the image acquisition information IAI and selecting and processing suited image data from the medical image MI.

In particular, this may involve determining appropriate displaying settings for the representation RE (optional sub-step I31). For instance, the displaying settings may comprise a contrast, brightness, intensity window (e.g., for lung or bone), viewing angle, image enhancement, cinematic rendering parameters and the like.

The displaying settings may be preconfigured and assigned to certain diagnostic use cases. As the image acquisition information IAI may indicate the diagnostic use case, it becomes possible to determine the displaying setting based on the image acquisition information IAI.

At step I40, the representation(s) RE generated in step I30 are displayed to the user U in the user interface 10. This may comprise generating appropriate control signals for operating the user interface 10 to display the representation(s) RE.

Optionally, the representation(s) RE may be generated according to a hanging protocol HP. The hanging protocol HP may define what kind of representation(s) RE are to be displayed and where they are to be displayed in a graphical user interface GUI. Further, the hanging protocol HP may set out subsequent processing steps such as the retrieval of comparative images CI or the application of image processing tools IPT. At step I41, a preconfigured hanging protocol HP may be retrieved from the hanging protocol database HPDB which matches the image acquisition information IAI. To this end, a lookup operation may be performed in the hanging protocol database HPDB for a hanging protocol HP corresponding to the image acquisition information IAI. Specifically, an association linking the hanging protocols HP in the hanging protocol database HPDB with image acquisition information IAI or corresponding use-cases may be used to find suitable hanging protocols HP.

At optional step I50, a comparative medical image CI may be retrieved and provided alongside the medical image MI. A comparative medical image CI may generally relate to a medical image which is helpful for arriving at a medical diagnosis based on the medical image MI. AS such, the comparative medical image CI may relate to a prior study of the patient. As an alternative, the comparative medical image CI may be a similar image of different patient which may already have been diagnosed. Further, the comparative medical image CI may be an excerpt from an electronic compendium such as an electronic textbook. The type of the comparative medical image CI may be defined in the hanging protocol HP or linked to the diagnostic use case respectively identified based on the image acquisition information IAI.

Specifically, at sub-step I51, the comparative medical image CI may be retrieved from the database DB. Thereby, medical images may be retrieved which match the image acquisition information IAI. For instance, if the image acquisition information IAI indicates that a certain body part was imaged based on a HASTE sequence, the database DB may be searched for medical images which relate to the same body part and at least comparable sequences. To draw such comparison, the medical images of a patient in the database coming into question may be subjected to the same processing as the medical image MI for deriving an image acquisition information IAI.

At sub-step I52, the comparative medical image CI may be subjected to an appropriate image processing for preparing a comparative representation CRE therefrom which can be readily compared to the representation RE of the medical image MI. In particular the same image processing may be applied to the comparative medica image CI which was used for the medical image MI. In particular, the same display settings may be used.

At sub-step I53, the comparative representation CRE is displayed together with the representation RE. Specifically, the comparative representation CRE may be displayed according to the hanging protocol HP selected at step I41 in the graphical user interface GUI.

At optional step I60, an image processing result FI may be generated based on the medial image MI and provided to the user U via the graphical user interface GUI. The image processing result may be generated according to the image acquisition information IAI and/or the hanging protocol HP. The image processing result FI may relate to a medical finding, a measurement or a segmentation extracted from the medical image MI. The image processing result FI may be generated using a corresponding image processing algorithm or tool IPT which may be executed by the computing unit.

Specifically, at sub-step I61, an image processing tool IPT may be selected from the tool database TDB according to the image acquisition information IAI (or according to the diagnostic use case and/or hanging protocol HP respectively identified based on the image acquisition information IAI). The look-up of the image processing tool IPT may be based on an association linking the image processing tools IPT in the tool database TDB with image acquisition information IAI (or hanging protocols HP or diagnostic use cases).

At sub-step I62, the selected image processing tool IPT may be applied to the medical image MI. Optionally, at sub-step I62, the image processing tool IPT may also be applied to any comparative medical image CI to obtain a comparative image processing result FI.

At sub-step I63, the image processing result FI is displayed to the user U in the graphical user interface GUI. The displaying may be in accordance with any rules in the selected hanging protocol HP specifying the arrangement of image processing results FI in the graphical user interface GUI.

At optional step I70, a reporting template RT on the basis of which a medical report may be completed by the user U may be selected and provided. The reporting template RT may be provided to the user U in the graphical user interface GUI. Thereby, the location of the reporting template RT may be determined by the hanging protocol HP.

Specifically, at step I71, a reporting template RT is retrieved from the report database RDB which matches the image acquisition information IAI. To this end, a lookup operation may be performed in the reporting database RDB for reporting template RT corresponding to the image acquisition information IAI. Specifically, an association linking the reporting templates RT with hanging protocols HP or diagnostic use cases (both of which may be identified based on the image acquisition information IAI) may be used to find correct reporting templates RT.

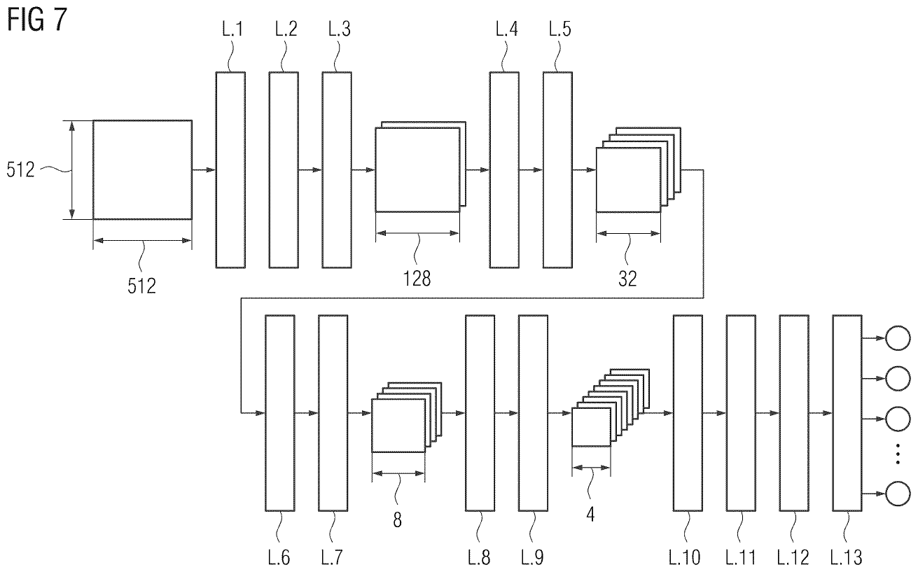

In FIG. 7, an embodiment of the trained function TF is displayed. In the example shown in FIG. 7, the trained function TF is a convolutional neural network, in particular, a deep convolutional neural network. Of note, this is just meant as an illustrative example as the trained function TF may also be embodied by any other suitable machine learned models such as transformer architectures or FocalNets as elsewhere herein described.

The trained function TF according to FIG. 7 comprises convolutional layers, pooling layers and fully connected layers. In the input layer L.1, there is one node for each pixel of the k-space image KI, each pixel having one channel (the respective intensity value). After the input layer, there are four convolutional layers L.2, L.4, L.6, L.8, each of the four convolutional layers is followed by a pooling layer L.3, L.5, L.7, L.9. For each of the convolutional layers, a 5×5 kernel is used (indicated by “K: 5×5”) with a padding of (indicated by “P:2”) and either one or two filters/convolutional kernels (indicated by “F:1” or “F:2”).

From the pooling layers L.3, L.5, L.7, L.9, the first three layers L.3, L.5, L.7 implement an averaging operation over patches of size 4×4, and the last pooling layer L.9 implements a maximum operation over patches of size 2×2. The additional layer L.10 of FIG. 7 flattens the input k-space images KI. However, this layer is not relevant for the actual calculation.

The last layers of the network are three fully connected layers L.11, L.12, L.13, the first fully connected layer having 128 input and 40 output nodes, the second fully connected layer L.12 having 40 input and 10 output nodes, and the third fully connected layer L.13 having 10 input and n output nodes, wherein the n output nodes form the output layer of the whole machine learning model.

The value of the first node of the output layer may correspond to one element of the image acquisition information IAI (e.g., MR or CT imaging procedure) of the medical image MI related to the input k-space image. The second node may relate to another element of the image acquisition information (e.g., spin echo sequence or gradient echo sequence) and so forth. There may be as many output nodes as elements in the image acquisition information IAI the trained function TF has to discriminate.

For training the trained function TF, a database of 500 medical images MI with confirmed image acquisition information IAI has been used. The database was split into training data (320 datasets), validation data (80 datasets) and test data (100 datasets). That followed, the medical images MI were transformed into k-space images KI. For training the trained function TF, the backpropagation algorithm was used based on a cost function L(x, y1, y2, . . . yn) =|M(x)1−y1|2+|M(x)2−y2|2+ . . . +|M(x)n−yn|2 wherein x denotes an input k-space image Kl, y1 denotes whether a first element of the image acquisition information IAI is indicated, y2 denotes whether a second element of the image acquisition information IAI is indicated, and yn denotes whether an n-th element of the image acquisition information IAI is indicated. Furthermore, M(x) denotes the result of applying the trained function TF to the input k-space image KI, and M(x)1, M(x)2, . . . ,M(x)n correspond to the value of the first, second, . . . n-th output node if applying the trained function TF to the input k-space image KI.

Based on the validation set of 80 datasets and the corresponding annotations, the best performing trained function TF out of several machine learning models (with different hyperparameters, e.g., number of layers, size and number of kernels, padding etc.) was selected. The specificity and the sensitivity were determined based on the test set comprising 100 datasets and the image acquisition information IAI.

Wherever meaningful, individual embodiments or their individual aspects and features can be combined or exchanged with one another without limiting or widening the scope of the present invention. Advantages which are described with respect to one embodiment of the present invention are, wherever applicable, also advantageous to other embodiments of the present invention. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A computer-implemented method for providing an image acquisition information of a medical image, the method comprising:
obtaining the medical image;
transforming the medical image into a frequency domain to obtain a k-space image;
determining the image acquisition information by applying a trained function to the k-space image; and
providing the image acquisition information.

2. The computer-implemented method of claim 1, wherein the image acquisition information comprises at least one of an image acquisition parameter or an information about a body part depicted in the medical image.

3. The computer-implemented method of claim 2, wherein
the medical image has been acquired with a magnetic resonance acquisition procedure using a magnetic resonance medical imaging modality, and
the image acquisition parameter relates to at least one of an image weighting or a magnetic resonance sequence used in the acquisition procedure.

4. The computer-implemented method of claim 1, wherein the determining includes applying the trained function on the medical image.

5. The computer-implemented method of claim 1, further comprising:
generating a merged image based on the medical image and the k-space image, wherein
the determining includes applying the trained function on the merged image.

6. A computer-implemented method for displaying a representation of a medical image, the method comprising:
receiving the medical image from a database;
determining an image acquisition information of the medical image according to the method of claim 5;
generating a representation of the medical image for displaying in a user interface based on the image acquisition information; and
displaying the representation in the user interface.

7. The computer-implemented method of claim 6, wherein
the generating the representation comprises selecting a displaying setting for generating the representation based on the image acquisition information and applying the selected displaying setting upon generating the representation,
the displaying setting including at least one of a contrast setting, a brightness, an intensity windowing, an image enhancement, a look-up table, a viewing plane, a segmentation mask, a zoom level or panning, or a volumetric rendering parameter.

8. The computer-implemented method of claim 6, further comprising:
selecting, based on the image acquisition information, a hanging protocol including a rule set for displaying one or more representations of an image in a user interface, wherein
the displaying displays the representation based on the hanging protocol.

9. The computer-implemented method of claim 6, further comprising:
retrieving, from the database, a comparative medical image based on the image acquisition information,
processing the comparative medical image to generate a comparative representation for displaying in the user interface; and
displaying the comparative representation in the user interface.

10. The computer-implemented method of claim 9, wherein
the medical image was acquired from a patient at a first point in time and the comparative medical image was acquired from the patient at a second point in time, the second point in time different than the first point in time.

11. The computer-implemented method of claim 6, further comprising:
selecting, based on the image acquisition information, an image processing tool configured to provide an image processing result;
apply the selected image processing tool to generate the image processing result; and
displaying the image processing result in the user interface.

12. The computer-implemented method of claim 6, further comprising:
selecting a reporting template for producing a medical report corresponding to the medical image based on the image acquisition information; and
providing the reporting template via the user interface.

13. A system for providing an image acquisition information of a medical image, the system comprising:
an interface unit; and
a computing unit, wherein the computing unit is configured to cause the system to receive the medical image via the interface unit,
transform the medical image into a frequency domain to obtain a k-space image, determine the image acquisition information by applying a trained function to the k-space image, and
provide the image acquisition information via the interface unit.

14. A system for providing control signals for displaying a representation of a medical image, the system comprising:
an interface unit; and
a computing unit, wherein the computing unit is configured to cause the system to, receive the medical image from a database via the interface unit,
transform the medical image into a frequency domain to obtain a k-space image, determine image acquisition information by applying a trained function to the k-space image,
generate control signals for controlling a user interface to display a representation of the medical image, the representation being adapted according to the image acquisition information, and
provide the control signals to the user interface via the interface unit.

15. A computer program product comprising program elements, when executed by a computing unit, cause the computing unit to perform the method of claim 1.

16. A non-transitory computer-readable medium on which program elements are stored that, when executed by a computing unit, cause the computing unit to perform the method of claim 1.

17. The computer-implemented method of claim 7, further comprising:
selecting, based on the image acquisition information, a hanging protocol including a rule set for displaying one or more representations of an image in a user interface, wherein
the displaying displays the representation based on the hanging protocol.

18. The computer-implemented method of claim 7, further comprising:
retrieving, from the database, a comparative medical image based on the image acquisition information,
processing the comparative medical image to generate a comparative representation for displaying in the user interface; and
displaying the comparative representation in the user interface.
